(19) United States
(12) Patent Application Publication
(10) Pub. No.: US 2006/0027467 A1
(43) Pub. Date: Feb. 9, 2006

(54) Anti-microbial suture material dispenser system

(76) Inventor: Patrick J. Ferguson, Portland, OR (US)

Correspondence Address: GLENN C. BROWN, PC, 777 NW WALL STREET, SUITE 308, BEND, OR, 97701, US

(21) Appl. No.: 10/912,648

(22) Filed: Aug. 4, 2004

(51) Int. Cl. A61B 17/06

(52) U.S. Cl. 206/63.3

(57) ABSTRACT

A suture-material-dispenser system for a supply of suture material includes the supply of such material and a housing which defines a cavity for containing the supply. The housing includes a top region that has an opening formed in it and an antimicrobial plug within the opening. The plug seals the cavity and has a suture-material-dispensing port to allow dispensing of suture material therethrough. The antimicrobial plug comprises silver, which is durable, non-reactive, cost-effective and has antimicrobial properties.

### BACKGROUND OF THE INVENTION

1. Technical Field

The present invention relates generally to antimicrobial suture-material dispensers. More particularly, the invention concerns a novel suture-material-dispenser system that comprises a silver-impregnated plug material.

2. Field of the Invention

Conventional suture dispensers are well known. Such dispensers are characterized by having relatively large dimensions, and are constructed for holding wet suture material, one type of conventional suture material that is packaged in an alcohol-based solution. The other type is dry suture material and it will be discussed below in connection with the present invention.

To sterilize wet suture material in conventional, so-called “wet-pack⇄ dispensers, an extreme procedure must be followed which involves irradiating the filled dispenser with gamma-ray radiation. Such a procedure is extreme because it tends to modify or adversely affect the molecular structure of the suture material.

After irradiation, suture material from conventional “wet packs” tends to become weaker and stiffer, which makes it more difficult for a surgeon to work with. The integrity of the material is also compromised. Such an extreme sterilization procedure is required because there is no other known way to sterilize wet suture material.

Conventional dispensers, or “wet packs”, are also disfavored because the wet suture material is messy, relatively heavy and flammable.

Conventional dispensers also require a two-handed operation for dispensing and cutting suture material. Essentially, the user, such as a surgeon or other surgical health care professional, holds the dispenser while removing a desired amount of suture material from the supply contained within the dispenser. Next, the user grasps a cutting instrument such as a pair of scissors and cuts the desired amount from the supply.

With respect to wet packs, conventional dispensers require about 2-4 ounces of sterilization liquid such as a solution of 90% isopropyl alcohol and 10% water. The current liquid-volume requirement of conventional dispensers results in an undesirably heavy package which is costly to ship or transport due to weight and hazardous material charges.

None of the conventional dispensers is designed for one-handed dispensing and cutting operation, and none is constructed for holding dry suture material.

Another problem with conventional dispensers is maintaining sterilization and the potential for bacterial growth.

### BRIEF SUMMARY OF THE INVENTION

Accordingly, it is a principal object of the present invention to provide a suture-material-dispenser system which overcomes the drawbacks of prior art systems.

Yet another object is to provide such a system that allows for a one-handed dispensing and cutting operation.

Another important object of the invention is to provide such a system with a flip-top for promoting one-hand operation of opening and closing.

Still another object is to provide such a system that is sized for easy storage and transport.

Yet another object is to provide such a system that is usable for wet or dry suture material.

Still another object is to provide such a system for holding wet suture material and an optimal amount of sterilization fluid.

A further object is to provide an antimicrobial plug such that the suture material passes through the antimicrobial plug upon exiting the dispenser.

It is also an object of the invention to provide such a system that can be cost-effectively manufactured.

In brief summary, one aspect of the invention includes a suture-material-dispenser system for a supply of dry or wet suture material with a housing and a gas-permeable, antimicrobial resilient plug. The housing defines a cavity for containing a supply of suture material, and it includes a top region that has an opening formed in it. The gas-permeable, antimicrobial resilient plug has a body that fits sealingly within the opening, and the body has formed in it a suture-material-dispensing port for allowing suture material to be dispensed therethrough. The port is preferably formed as a slit with a length of about 2-6 mm.

The plug body assumes a pre-dispense condition and a dispense condition, and the plug body is formed from a substance with a memory characteristic allowing that section of the body adjacent the port to deform when the body is in the dispense condition, thus to minimize degradation of suture material during dispensing operation. The memory characteristic also allows the body to return substantially to its undeformed state when the body is in it's pre-dispense condition, thus to seal substantially the cavity from contaminant.

The plug body is impregnated with silver, which has antimicrobial properties. As an antimicrobial, silver has been shown to be effective in killing over 650 strains of bacteria. In addition, silver is stable, non-reactive, cost-effective, durable and non-toxic. Silver has been shown to be safe for oral, internal and food-related applications. The antimicrobial properties of silver are long-lasting and are delivered consistently throughout the life of the product. Thus, the antimicrobial plug is effective in maintaining the sterility of the suture material dispenser and in preventing bacterial buildup around the opening in the suture material dispenser.

The invention preferably also includes the following other features. An anti-contaminant, flip-top cover is pivotably attached to the top region, and is constructed for releasable closure over the top region substantially to prevent contaminants from entering the cavity. The top region also includes a lip that extends outwardly from the housing, and the cover includes a downwardly extending expanse that is engageable with lip to obtain such releasable closure.

An on-board cutter is preferably attached to the housing adjacent the resilient plug for allowing the user to cut a desired dispensed amount of material from such supply. The antimicrobial, resilient plug is preferably formed from a material such as liquid-injection-molded silicone, which material is ethylene-oxide-gas permeable, and has the above-described memory characteristic. The resilient plug is also preferably formed from a material with a hardness in the range of about 40-80 on a Shore A durometer.

In addition, the plug material comprises silver. Preferably, the silver is in the form of silver ions in a zeolite carrier, such as that available from AgION, Technologies, LLC. The silver can be embedded into the plug body or coated onto the plug body.

Another aspect of the invention includes the above suture-material-dispenser system and the supply of dry suture material. That version of the invention also includes a reel fittable within the cavity, and having wound on it the supply. The housing also includes a bottom region and a semi-circular bearing positioned in the bottom region for supporting the reel.

Yet another aspect of the invention includes the above suture-material-dispenser system and a supply of wet suture material. The wet suture material is wound on a reel fittable within the cavity, and the wet suture material is wetted by an amount of sterilization liquid. The reel is sized to optimize the quantity of sterilization liquid disposed within the cavity.

These and other objects and advantages of the invention will be more clearly understood from a consideration of the accompanying drawings and the following description of the preferred embodiment.

### BRIEF DESCRIPTION OF THE DRAWINGS

The nature, principle and utility of the present invention will be clearly understood from the following detailed description when read in conjunction with the accompanying drawings, wherein:

FIG. 1 is an isometric view showing the preferred embodiment of the suture dispenser system of the present invention with its cover being pivoted to an open position allowing access to dry suture material contained in the system.

FIG. 2 is an enlarged, fragmentary, view of a top section of the system shown in FIG. 1 which illustrates the dispensing and cutting operations achievable using the invention.

FIG. 3A is an enlarged, fragmentary, cross-sectional view through line 3-3 of FIG. 1.

FIG. 3B is an enlarged, fragmentary, cross-sectional view like FIG. 3A, only showing in an exaggerated way how the resilient member of the present invention deforms during dispensing.

FIG. 4 is a side sectional view of the system shown in FIG. 1 after the cover is moved to a closed position.

FIG. 5 is an enlarged, fragmentary, cross-sectional view through line 3-3 of FIG. 1 showing an alternative embodiment of the plug.

FIG. 6 is an isometric view of an alternative embodiment of the housing of the present invention, shown in a position similar to that shown in FIG. 1.

FIG. 7 is a side view of the housing shown in FIG. 6, shown in approximately actual size.

FIG. 8 is an end view of the housing shown in FIG. 7, shown on about the same scale as FIG. 7.

FIG. 9 is a top view of the housing shown in FIG. 7, shown on about the same scale as FIG. 7.

FIG. 10 is an enlarged view of the surface of the plug body and the mechanism that occurs when the plug body comes into contact with the ambient.

The drawings are for illustrative purposes only and are not drawn to scale. In the drawings, the same numbers are used for the same part or portion throughout the drawings.

### DETAILED DESCRIPTION OF THE INVENTION

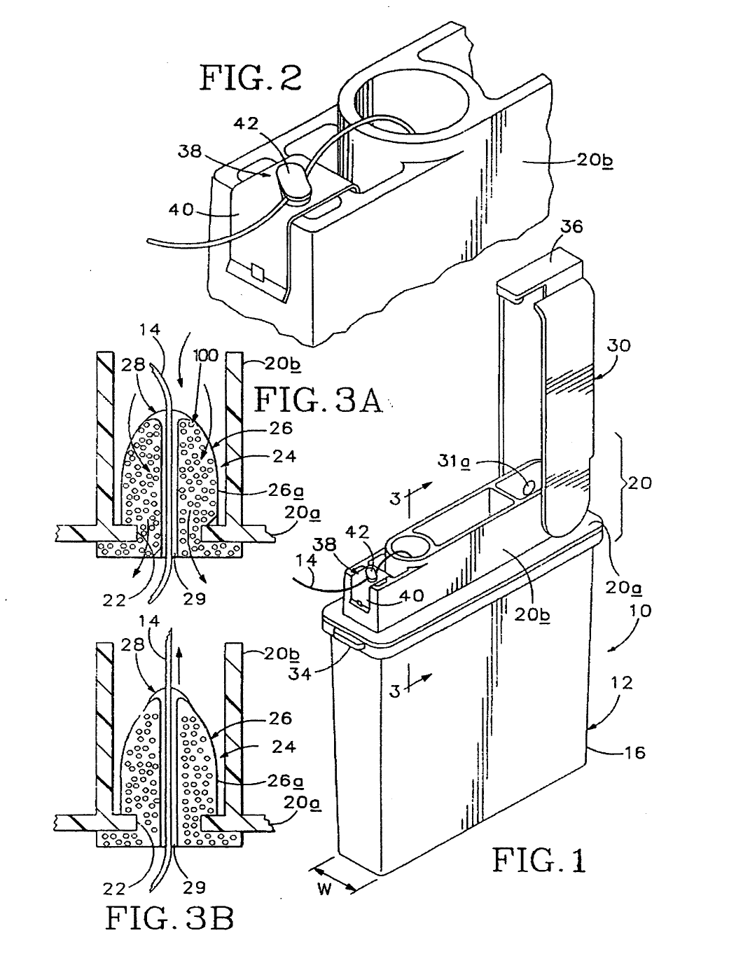

FIG. 1 depicts an isometric view of the suture dispenser system of the present invention, being made in accordance with its preferred embodiment and indicated at 10. The preferred embodiment includes the to-be-described dispenser 12 and dry-suture material (“DSM”) 14. DSM 14 can be obtained from CP Medical of Portland, Oreg. and suitable versions of DSM 14 are sold by CP Medical under the trademarks FLEX-GUT, MONOMID, and POLYAMID.

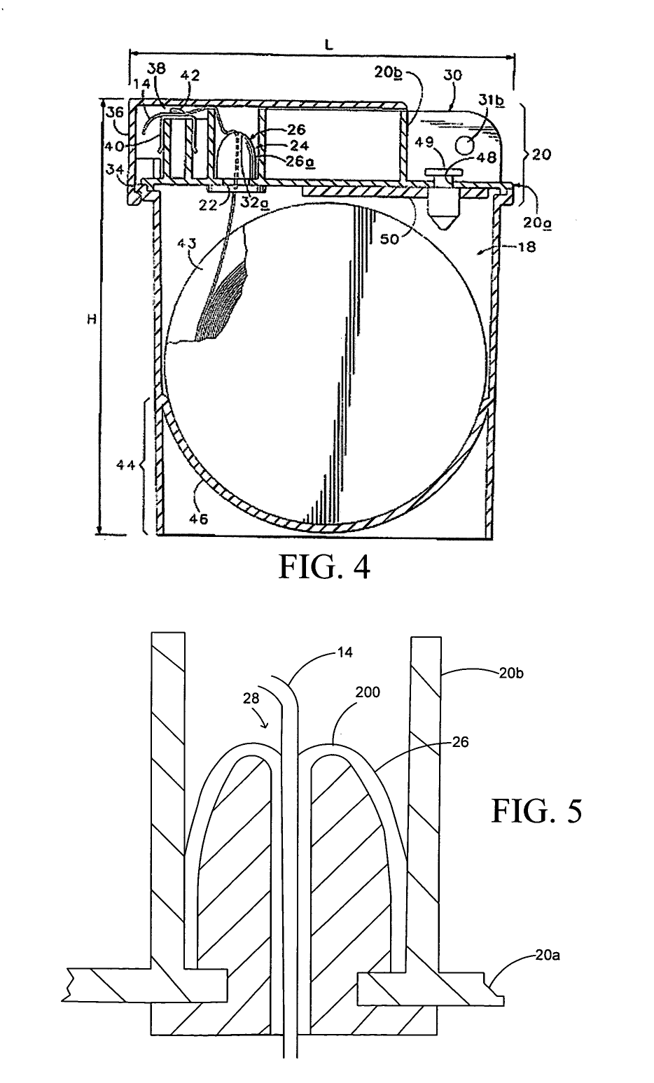

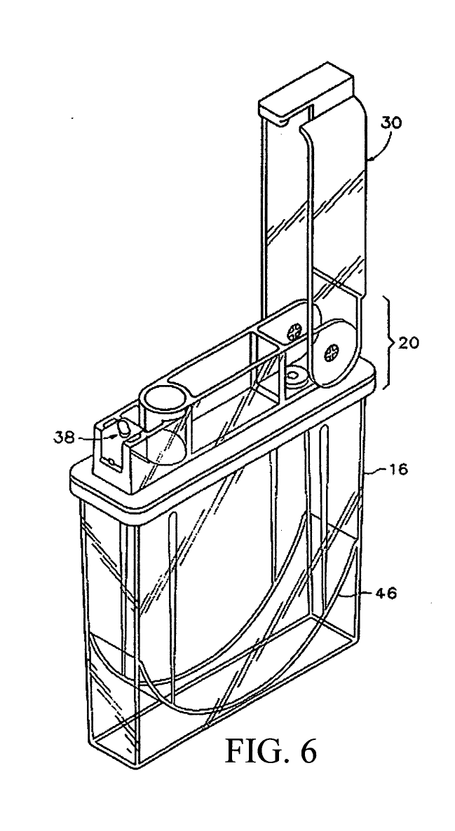

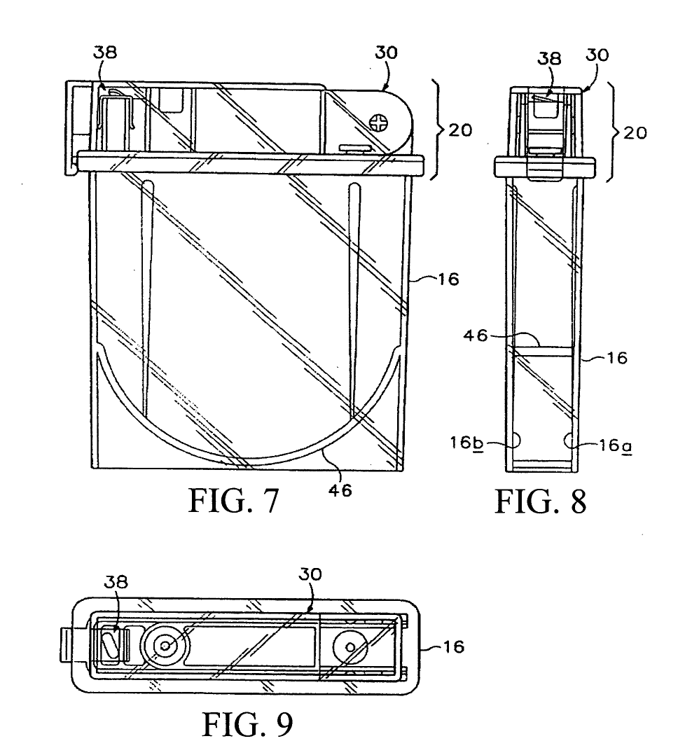

Referring to FIGS. 1, 4 and 8, the invention includes a housing 16, preferably made from clear ektar copolyester, which defines a cavity 18 (FIG. 4) for containing a supply of DSM 14. The housing includes opposing walls 16a-b (FIG. 8) and a top region 20 that has an opening 22 (FIG. 4) formed in it. Fitting sealingly within opening 22 is a gas-permeable, resilient antimicrobial plug 24 having a body 26 with a suture-material-dispensing port 28 (FIGS. 3A and 3B) for allowing DSM 14 to be dispensed through it. Top region 20 includes a plate-like member 20a which can be fastened to housing 16 using suitable means such as adhesive. All components of system 10 are preferably made of a suitable plastic, but any suitable material may be used. Top region 20 also includes a raised member 20b which is positioned fixedly and centrally of plate-like member 20a.

With respect to material choice for body 26, the preferred material is liquid-injection-molded, or LIM, silicone or a polymeric material. The material also has a hardness in the range of about 40-80 on a Shore A durometer. The material is also preferably ethylene-oxide-gas permeable to allow system 10 to be sterilized according to a conventional ethylene-oxide-gas sterilization procedure.

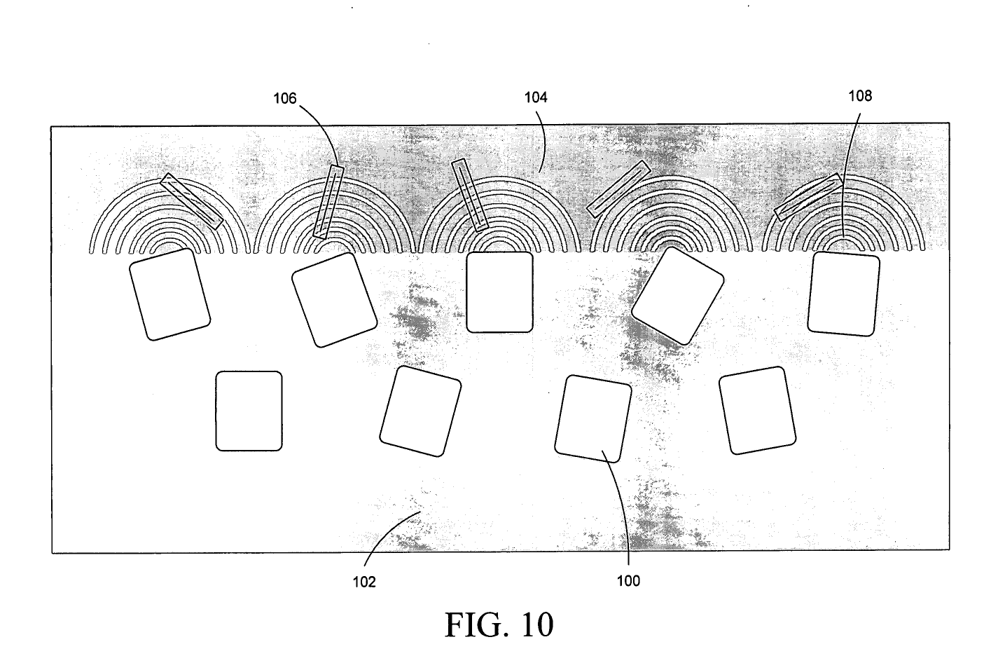

In addition, the plug body 26 is impregnated with silver material 100, which provides the antimicrobial properties of the plug. Preferably, the silver material 100 is an antimicrobial compound comprising silver ions on a zeolite carrier, such as the compound available from AgION Technologies, LLC. Silver ions are a naturally-occurring microbe inhibitor. The antimicrobial compound allows a controlled and effective release of silver ions, thereby preventing growth and migration of bacteria, yeast, mold and fungus. Essentially, an ion exchange release mechanism occurs wherein the silver ions in the antimicrobial compound are exchanged with sodium or other ions present in the atmosphere, thus causing a controlled release of silver on demand. FIG. 10 is a graphical representation of this mechanism. As shown in FIG. 10, the antimicrobial compound 100 is dispersed in the plug material 102. The surface film of moisture 104 contains bacteria 106. When the bacteria 106 comes into contact with the antimicrobial plug, ion release occurs, depicted by the semi-circular ripples 108.

Since silver is substantially inert, it will not react with the plug material or the suture material and cause degradation over time. Thus, the antimicrobial compound has little impact upon the performance of the suture dispenser and does not cause major processing challenges in the manufacture of the suture dispenser. Additionally, because of the cost-effectiveness of the compound, it will add very little to the cost of manufacturing the suture dispenser. In fact, it has been estimated that adding the antimicrobial compound to toothpaste, for example, would add about 0.5 cents per ounce to the cost of the toothpaste.

The antimicrobial compound is also very durable and can withstand temperatures up to 800° C. and pH values between 3 and 10 while retaining full antimicrobial effectiveness. In addition, the antimicrobial compound has been shown to retain its antimicrobial properties for the life of the product without causing antibiotic resistance. In practice, the antimicrobial compound releases silver ions at a steady rate for 25 to 30 years and it is estimated that, theoretically, the antimicrobial properties could last for about 170 years.

The antimicrobial compound has been tested for biocompatibility and shown to be non-toxic. The compound has passed ISO-10993-1 biocompatibility testing and has been shown to be safer than ordinary table salt. The antimicrobial compound has been tested and marketed in health, apparel and child care products and proven safe for oral, internal and food-related applications.

Referring to FIGS. 3A-3B, two conditions of body 26 will be described, a pre-dispense condition and a dispense condition. These figures will illustrate a to-be-described memory characteristic of body 26. FIG. 3B shows, in an enlarged and exaggerated way, that the portion of body 26 adjacent port 28 deforms when the body is in the dispense condition with DSM 14 being pulled out of cavity 18 (FIG. 4) in the direction of the arrow. Such deformation, combined with the to-be-described softness of body 26, tends to minimize degradation of suture material during dispensing operations.

Referring back to FIG. 3A, the memory characteristic of body 26 allows the body to return substantially to its undeformed state when the body is in its pre-dispense condition as shown. The pre-dispense condition occurs when no dispensing, or pulling, force is applied to DSM 14. By returning to its undeformed state, body 26 will seal substantially cavity 18 (FIG. 4) from contaminant. Preferably, port 28 is formed as a slit, with a length of about 2-6 mm, in the top surface 26a of body 26 to maximize the capability of body 26 to seal cavity 18 from contaminant. Body 26 is shown in FIGS. 3A-3B also with a cylindrical void 29 which communicates with port 28 and cavity 18. The shape of that void is not critical however, and body 26 could also be constructed substantially hollow (undepicted) with only a suitable uniform thickness associated with its surface area.

Still referring to FIG. 3A, there is also depicted the gas-permeable nature of the plug 24 as sterilizing gas, shown by arrows, penetrates through body 26 into cavity 18.

Referring to FIGS. 1 and 4, system 10 also preferably includes an anti-contaminant, flip-top cover 30 which is pivotably attached to top region 20 via opposing bosses 31a-b which extend inwardly from opposing sides of the cover into suitable, corresponding holes formed in top region 20. Cover 30 is constructed for releasable closure over the top region substantially to prevent contaminants from entering the cavity. In this way the cover acts as a backup seal to the primary seal provided by the plug body 26 as described above in connection with FIG. 3A.

To provide releasable closure of cover 30 over top region 20, top region 20 is preferably constructed with a lip 34 that extends outwardly from housing 16, and cover 30 includes a downwardly extending expanse 36 that is engageable with lip 34 to obtain such releasable closure.

Referring to FIGS. 1-2 and 4, system 10 also preferably includes an on-board cutter 38 attached to raised member 20b of top region 20 of housing 16 adjacent resilient member 24. Cutter 38 allows the user to cut a desired dispensed amount of DSM 14 from the remaining supply of that material in cavity 18. Cutter 38 is preferably made as an elongate section 40 of a suitable metal. Section 40 is die cut to form a cutter blade 42 which is bent upwardly a suitable amount to allow DSM 14 to be fed under it and, ultimately, pulled against it to cut a desired amount of DSM 14 from the supply. Section 40 is formed as a clip to fit over a section of raised member 20b, but any suitable means of attaching cutter 38 to dispenser 12 may of course be used.

Referring to FIG. 4, system 10 also preferably includes a reel 43 fittable within cavity 18 for retaining the supply of DSM 14. To support reel 43, a bottom region 44 of housing 16 includes a semi-circular bearing 46 positioned in it. Given the below described dimensions of system 10, reel 43 is sized to hold between about 50-110 yards of DSM 14.

Referring to FIG. 4, there is shown an auxiliary opening 48 formed in top region 20 with a conventional nipple 49 sealing positioned therein. A support plate 50 is also suitably attached to the underside of plate-like member 20a for supporting nipple 49. With nipple 49, the system of the present invention can also be used with wet-suture material (“WSM”), and nipple 49 can be used as what is known as a fill hole for adding wet sterilization liquid such as the usual alcohol-based ones.

FIG. 5 shows an alternative embodiment of the plug body 26. The plug body 26 is coated with a layer of antimicrobial material 200. The antimicrobial material 200 is preferably in the form of silver ions in a zeolite carrier.

In use, system 10 can be held easily in the user's hand and stored in a pocket of the user's clothing. Referring to FIGS. 1 and 4, the overall dimensions of system 10 are about 4 5/16″ high (“H”)×3 13/16″ long (“L”)×⅞″ wide (“W”). From its covered position as shown in FIG. 4, the user simply presses outwardly and upwardly against the bottom of expanse 36 (i.e. the bottom as shown in FIG. 4) to disengage it from lip 34 and open cover 30. When cover 30 is in its open position (FIG. 1), the user simply pulls a desired amount of DSM 14 from housing 16 and cuts that amount from the supply of DSM 14 by feeding under cutter blade 42 and then pulling that amount against that blade.

After dispensing, the user is able to close flip-top cover 30 with the same hand being used to hold system 10 by simply pushing down on an accessible section of the cover with the thumb. With respect to later use of the section of DSM 14 that extends outwardly from resilient member 24 to cutter 38 (see FIG. 4), it will depend on the user's own sterile procedures. If sterile gloves are used when handling DSM 14, it is likely that that section is sufficiently sterile for use.

It should now be understood that the present invention meets the above objects by providing a suture-material-dispenser system which overcomes the drawbacks of prior art systems. System 10 is constructed for holding and dispensing dry suture material, and for maintaining the sterility of the suture material dispenser and preventing bacterial buildup around the opening in the suture material dispenser. System 10 also includes a flip-top for promoting one-hand operation of opening and closing. Cutter 38 makes it possible to perform one-handed dispensing and cutting. The overall dimensions of system 10 make it easy to handle, store and transport on the user's person. Because system 10 also includes nipple 49, it can be used with wet or dry suture material. Also, system 10 can be cost-effectively manufactured.

Accordingly, while a preferred embodiment of the invention has been described herein, it is appreciated that modifications are possible that are within the scope of the invention.

What is claimed is:

1. A suture-material-dispenser system comprising:
a housing which defines a cavity for containing suture material, with the housing including a top region that has an opening; and
a plug having a body that fits sealingly within the opening and the body having a suture-material-dispensing port for allowing the suture material to be dispensed therethrough,
wherein the plug comprises an antimicrobial material.

2. The suture-material-dispenser system of claim 1, wherein the antimicrobial material comprises silver.

3. The suture-material-dispenser system of claim 1, wherein the antimicrobial material is embedded in the body.

4. The suture-material-dispenser system of claim 1, wherein the antimicrobial material is coated on the body.

5. The suture-material-dispenser system of claim 1, wherein the antimicrobial material comprises silver ions on a zeolite carrier.

6. The suture-material-dispenser system of claim 1 wherein the body of the plug is formed from liquid- injection-molded silicone.

7. The suture-material-dispenser system of claim 1 wherein the body of the plug is formed from polymeric material.

8. The suture-material-dispenser system of claim 5 wherein the antimicrobial material provides a controlled release of the silver ions.
